(19) United States
(12) Patent Application Publication
(10) Pub. No.: US 2021/0077454 A1
(43) Pub. Date: Mar. 18, 2021

(54) TRANSFORMATION OF CANNABINOL AND TERPENE OILS INTO WATER SOLUBLE DRY POWDERS FOR SOLID FORM SUBLINGUAL DELIVERY

(71) Applicant: Pure Green Pharmaceuticals, Inc., West Bloomfield, MI (US)

(72) Inventors: John Althaus, Saline, MI (US); Stephen Goldner, West Bloomfield, MI (US)

(21) Appl. No.: 16/961,432

(22) PCT Filed: Jan. 11, 2019
(86) PCT No.: PCT/US2019/013126

§ 371 Date: Jul. 10, 2020

Related U.S. Application Data
(60) Provisional application No. 62/617,206, filed on Jan. 13, 2018.

(51) Int. Cl. A61K 31/352; A61K 47/40; A61K 31/05

(52) U.S. Cl. CPC A61K 31/352; A61K 31/05; A61K 47/40

(57) ABSTRACT

A composition includes a clathrate compound that has guest molecules and carrier molecules that trap the guest molecules. The carrier molecules are a saccharide and the guest molecules include at least one of a cannabinoid or a terpene. The clathrate compound includes, by combined weight of the carrier molecules and the guest molecules, at least 18% of the guest molecules.

### CROSS-REFERENCE TO RELATED APPLICATIONS

This disclosure claims priority to United States Provisional Patent Application No. 62/617,206 filed on Jan. 13, 2018.

### BACKGROUND

Among other purposes, cannabis is used for medicinal reasons to treat disease or alleviate symptoms. For example, the active chemicals in medicinal cannabis can be delivered into the body through inhalation, ingestion, or topical application.

Delivery of medicinal cannabis through mucosal membranes is of particular interest due to rapidity of uptake without the detrimental effects associated with inhalation. In this regard, cannabinoids have been combined, to a limited degree, with specific cyclodextrins to produce a complex that is intended for sublingual or buccal administration.

### SUMMARY

A composition according to an example of the present disclosure includes a clathrate compound that has guest molecules and carrier molecules that trap the guest molecules. The carrier molecules are a saccharide and the guest molecules include at least one of a cannabinoid or a terpene. The clathrate compound has, by combined weight of the carrier molecules and the guest molecules, at least 18% of the guest molecules.

In a further embodiment of any of the foregoing embodiments, the saccharide is cyclic.

In a further embodiment of any of the foregoing embodiments, the saccharide is linear.

In a further embodiment of any of the foregoing embodiments, the carrier molecules include beta cyclodextrin.

In a further embodiment of any of the foregoing embodiments, the carrier molecules include 2-hydroxypropyl-beta-cyclodextrin.

In a further embodiment of any of the foregoing embodiments, the guest molecules include the terpene.

In a further embodiment of any of the foregoing embodiments, the guest molecules include the cannabinoid.

In a further embodiment of any of the foregoing embodiments, the carrier molecules include beta cyclodextrin and the guest molecules include the cannabinoid.

In a further embodiment of any of the foregoing embodiments, cannabinoid is selected from the group consisting of tetrahydrocannabinol, cannabidiol, cannabinol, cannabavarin, cannabigerol, cannabichromene, delta-8-THC, cannabicyclol, cannabitriol, cannabielsoin, and combinations thereof.

In a further embodiment of any of the foregoing embodiments, the clathrate compound includes, by combined weight of the carrier molecules and the guest molecules, up to 40% of the guest molecules.

A method of fabricating a clathrate compound according to an example of the present disclosure includes providing a liquid solution of a first solvent and a second, different solvent, and providing carrier molecules which are soluble in the first solvent and guest molecules which are soluble in the second solvent and insoluble in the first solvent. The carrier molecules are a saccharide and the guest molecules include at least one of a cannabinoid or a terpene. A first amount of the carrier molecules and a second amount of the guest molecules that is at least 1 molar equivalent to the first amount are then dissolved in the liquid solution. The concentration of the second solvent in the liquid solution is then decreased to cause the guest molecules to form a clathrate compound with the carrier molecules in which the carrier molecules trap the guest molecules.

A further embodiment of any of the foregoing embodiments includes, after the decreasing of the concentration, evaporating the liquid solution to produce a dry clathrate compound.

In a further embodiment of any of the foregoing embodiments, the second solvent is alcohol.

In a further embodiment of any of the foregoing embodiments, the first solvent is water.

In a further embodiment of any of the foregoing embodiments, the liquid solution contains, by volume, at least 90% of propanol.

In a further embodiment of any of the foregoing embodiments, the carrier molecules include beta cyclodextrin.

In a further embodiment of any of the foregoing embodiments, the decreasing of the concentration involves heating the liquid solution to a temperature above the boiling point of the second solvent.

A tablet according to an example of the present disclosure includes one or more excipients and a powder containing a clathrate compound that has guest molecules and carrier molecules that trap the guest molecules. The carrier molecules are a saccharide and the guest molecules include at least one of a cannabinoid or a terpene. The clathrate compound has, by combined weight of the carrier molecules and the guest molecules, at least 18% of the guest molecules.

In a further embodiment of any of the foregoing embodiments, the carrier molecules include beta cyclodextrin and the guest molecules include the cannabinoid.

In a further embodiment of any of the foregoing embodiments, cannabinoid is selected from the group consisting of tetrahydrocannabinol, cannabidiol, cannabinol, cannabavarin, cannabigerol, cannabichromene, delta-8-THC, cannabicyclol, cannabitriol, cannabielsoin, and combinations thereof.

### BRIEF DESCRIPTION OF THE DRAWINGS

The various features and advantages of the present disclosure will become apparent to those skilled in the art from the following detailed description. The drawings that accompany the detailed description can be briefly described as follows.

FIG. 1 illustrates a representative example of a carrier molecule shown as a chemical structure.

FIG. 2 illustrates the carrier molecule as a pictograph.

FIG. 3 illustrates a representative example of a guest molecule shown as a chemical structure.

FIG. 4 illustrates the guest molecule as a pictograph.

FIGS. 5 illustrates solubility of a carrier molecule.

FIG. 6 illustrates solubility of a guest molecule.

FIG. 7 illustrates an example method of fabricating a clathrate compound.

FIG. 8 illustrates a dry clathrate compound that is soluble in water or other aqueous solution.

FIG. 9 illustrates compounding of a clathrate compound with one or more excipients into a tablet.

FIG. 10 illustrates the water soluble properties of a tablet with a clathrate compound that is designed for sublingual release.

FIG. 11 illustrates the release of a clathrate compound into saliva of the oral cavity.

FIG. 12 illustrates a biochemical reaction in which a guest molecule is released from a clathrate compound.

FIG. 13 illustrates transmucosal delivery of a hydrophobic guest molecule to the blood stream.

### DETAILED DESCRIPTION

Disclosed herein, inter alia, is a composition of a clathrate compound, method of producing the clathrate compound, and products and treatments related thereto, for delivering pharmaceutically effective amounts of cannabis-based chemicals, particularly via sublingual delivery. Sublingual delivery effectiveness depends on the water-solubility of the chemicals being delivered. This poses challenges for sublingual delivery of cannabis-based chemicals, which are typically in the form of oils that are only very slightly soluble in water or are practically insoluble in water. For instance, viscous liquid cannabinoids and cannabis terpene oils cannot be readily absorbed sublingually and, regardless, are difficult to even incorporate into solid stable tablets for sublingual administration. In this regard, as will be appreciated from the present disclosure, the disclosed method, composition, products, and treatments are directed to a clathrate compound that has enhanced water-solubility and contains a high amount cannabinoid or terpene to enhance effectiveness.

The composition of the disclosed clathrate compound includes guest molecules and carrier molecules that trap or physically contain the guest molecules inside the carrier molecules. General configurations of clathrates is understood and is thus not discussed further herein. The guest molecules are cannabinoids or terpenes, and the carrier molecules are saccharides. The guest molecules are only slightly soluble in water or are practically insoluble in water. The carrier molecules may be soluble, freely soluble, or very soluble in water. “Soluble” used herein may refer to being soluble, freely soluble, or very soluble in water-terms which are well-understood in the chemical and pharmaceutical industry. The clathrate compound is at least soluble in water from the presence of the carrier molecules and serve to carry the cannabinoids or terpenes into solution with water. Further, due to the unique fabrication method disclosed herein, each carrier molecule may trap one or more guest molecules, which yields a clathrate compound that has relatively high amounts of the cannabinoid or terpene that heretofore have not been obtained, such as at least 18% or more by weight.

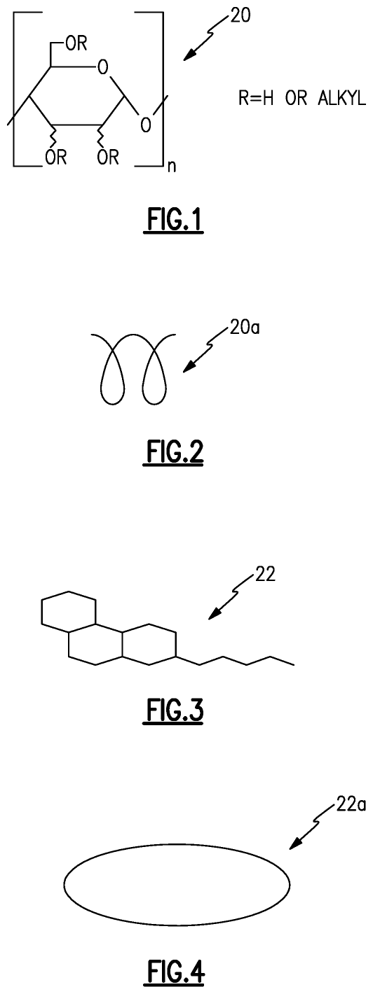

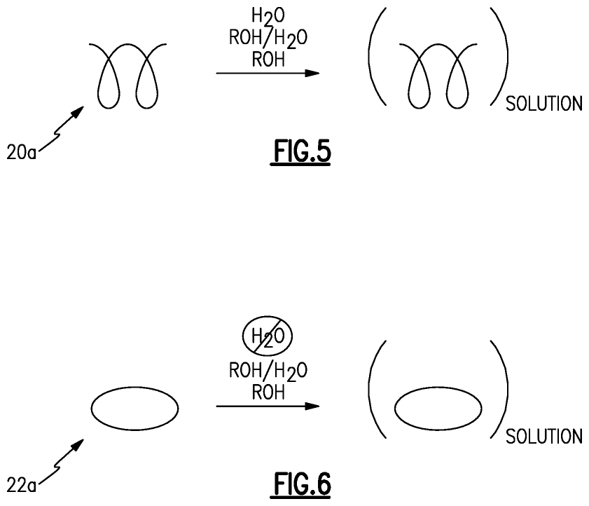

FIG. 1 illustrates a representative example carrier molecule 20 (shown as a chemical structure). FIG. 2 illustrates the carrier molecule as a pictograph 20a, which will be used in subsequent figures for simplicity. The carrier molecules 20 are saccharides and are of a general structure of an oligosaccharide as a polymer made from one or more types of monosaccharides. The monomer and length of the polymer may be of indeterminate identity and length. In one example, the monomer is a saccharide in which R groups are hydrogen (H) or akyl groups. The alkyl group can be a hydrocarbon which may or may not contain unspecified substituents. In one example, the polymeric structure is a linear, open-ended chain. The linear chain forms a helical secondary structure, which provides a cavity to trap the guest molecule. Alternatively, the saccharide has a cyclized structure in which the center of the ring serves as the cavity to trap the guest molecule. For example, the saccharide is a polymer of limited length in which terminal ends are attached. In one example, the carrier molecules are beta-cyclodextrin, such as but not limited to, 2-hydroxypropyl-beta-cyclodextrin (HPCD), hydroxyethyl-beta-cyclodextrin and methyl-beta-cyclodextrin.

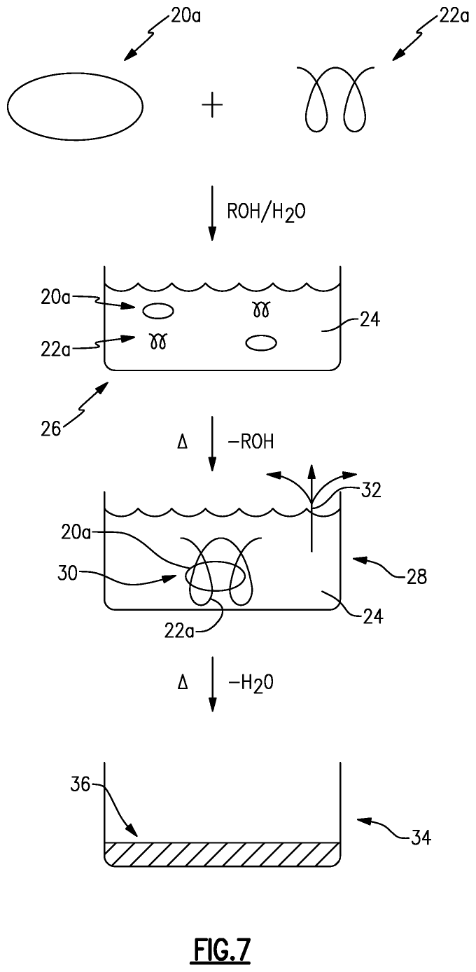

FIG. 3 illustrates a representative example of a guest molecule 22 (shown as a chemical structure). FIG. 4 illustrates the guest molecule as a pictograph 22a, which will be used in subsequent figures for simplicity. The guest molecules are selected from cannabinoids and terpenes, which are generally hydrocarbons. The cannabinoids can include, but are not limited to, tetrahydrocannabinol (delta-g-tetrahydrocannabinol, commonly known as “THC”), cannabidiol, cannabinol, cannabavarin, cannabigerol, cannabichromene, delta-8-THC, cannabicyclol, cannabitriol, and cannabielsoin. The terpenes can include, but are not limited to, myrcene, limonene, caryophyllene, linalool, and pinene.

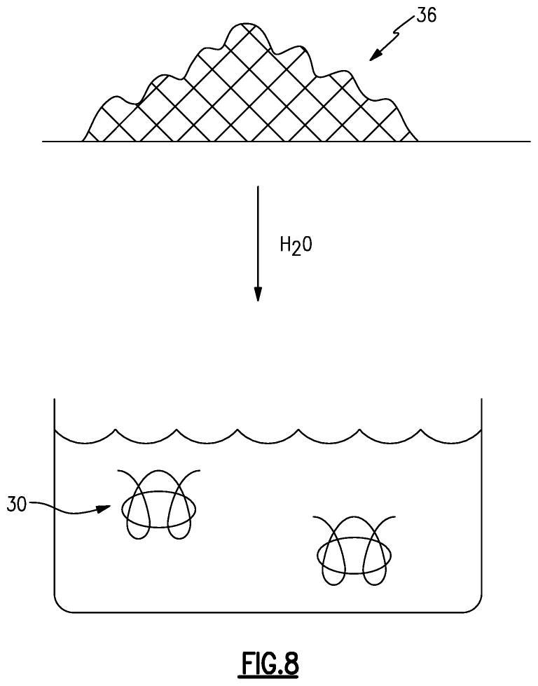

FIGS. 5 and 6 illustrate the general solubilities of the carrier molecule 20a and the guest molecule 22a, respectively. The carrier molecule 20a is at least soluble in polar liquid solutions such as water, water/alcohol, or alcohol. The guest molecule 22a is at least soluble in liquid solutions such as alcohol/water or alcohol but is only very slightly soluble or practically insoluble in water. Unless stated otherwise, any solubility represented herein is understood to be at normal temperature and pressure (NTP), which is 20° C. and 1 atm. As can be appreciated, both the carrier molecule 20a and the guest molecule 22a may be at least soluble in a solution of alcohol/water or alcohol.

FIG. 7 illustrates an example method of fabricating the clathrate compound. In general, the method involves dissolving both the carrier molecules 20a and the guest molecules 22a into a mutual solvent solution and then altering the concentration of the solution solvents to drive the guest molecules 22a into the carrier molecules 20a to form the clathrate compound.

The method may include first providing the carrier molecules 20a, the guest molecules 22a, and a liquid solution 24 in which both the carrier molecules 20a and the guest molecules 22a are mutually soluble. As an example, the liquid solution contains at least two solvents, such as a first solvent and a second, different solvent. The carrier molecules 20a are soluble in at least the first solvent. The guest molecules 22a are soluble in the second solvent but not the first solvent.

As an example, the first solvent is water and the second solvent is alcohol, such as isopropanol. Generally, it is preferable to use a liquid solution in which the concentration, by volume percentage, of the second solvent is greater than the concentration of the first solvent in order to ensure dissolution of the guest molecules 22a. The “providing” referred to herein may refer to furnishing the molecules 20a/22a and liquid solution 24 as starting materials and/or preparing the molecules 20a/22a and liquid solution 24 from precursor constituents for use as the starting materials.

As depicted at 26 in FIG. 7, the next step in the method involves dissolving the carrier molecules 20a and the guest molecules 22a in the liquid solution 24. As an example, a first amount of the carrier molecules 20a and a second amount of the guest molecules 22a are provided into the liquid solution 24. The liquid solution 24 may be stirred during and/or after the addition of the carrier molecules 20a and guest molecules 22a. For instance, the second amount of the guest molecules 22a is 1 molar equivalent or greater of the first amount of the carrier molecules 20a. In one example, 450 mg of THC is combined with 2,100 mg of HPCD using 50 ml of 91% isopropanol (9% water) as the liquid solvent 24 in a glass container. As will be appreciated with the benefit of this disclosure, the ratio of the amounts may be varied to control the amount of the cannabinoid or terpene in the final clathrate compound, subject to the efficiency of the guest molecules 22a forming the clathrate.

As depicted at 28 in FIG. 7, the next step in the method involves decreasing the concentration of the second solvent in the liquid solution 24. The decrease in concentration drives the guest molecules 22a to form the clathrate compound 30 with the carrier molecules 20a in which the carrier molecules 20a trap the guest molecules 22a. As an example, the concentration is gradually decreased by removing the second solvent as represented at 32, such as by evaporation, from the liquid solution 24.

In one example based on a liquid solution 24 of water as the first solvent and isopropanol as the second solvent, a container with the liquid solution is placed in an oven or other drying equipment and heated to approximately 110° C. (230° F.). The isopropanol evaporates and, as a result, the relative concentration of isopropanol in the liquid solution decreases and the relative concentration of water in the liquid solution increases. As the concentration of isopropanol decreases the guest molecules 22a reach a level of insolvency and the alcohol vacates the hydrophobic cavity inside the carrier molecules 20a. These events drive the guest molecules 22a to dynamically deposit into the cavity of the carrier molecules 20a to achieve greater stability, and thereby form the clathrate compound 30. Further, the relatively high mobility of the constituents in the liquid solution 24 enables substantially all of the carrier molecules 20a to receive one or more guest molecules 22a, depending on the molecular weight size of the selected guest molecules 22a, which results in a clathrate compound 30 having at least 18% by weight of the guest molecules 22a. Low-mobility systems and processes, such as freeze-drying, would be expected to be much less efficient in driving guest molecules into the carrier molecules to form the clathrate.

As depicted at 34 in FIG. 7, after the decreasing of the concentration, the remainder of the liquid solution 24 is evaporated to produce a dry clathrate compound 36. For instance, when most of the alcohol has evaporated, water is next to evaporate. At least a portion of the water may also evaporate with the alcohol. Once the alcohol and water fully evaporate, a powdery residue of the dry clathrate compound 36 remains. The residue may then be removed from that container for further processing, such as by scrapping using a straight edge razor blade or other similar tool. In instances where volatile cannabinoids or terpenes are used that may evaporate under the applied heat, the process can be modified to use a vacuum to induce evaporation instead of applied heat or in conjunction with lower applied heat.

The steps above produce a complex or clathrate compound between the carrier molecules 20a and the guest molecules 22a. The utilization of the decrease in the solvent concentration that changes from high non-polarity to high polarity that is produce by increased temperature allows for alcohol molecules in the hydrophobic pocket of the carrier molecules 20a to driven out by heat and replaced by the guest molecules 22a. This leads to relatively high efficiency in forming the clathrate compound, in which each carrier molecule 20a may contain at least one guest molecule 22a. As a result, the clathrate compound includes, by combined weight of the carrier molecules 20a and the guest molecules 22a, at least 18% of the guest molecules 22a and may include up to about 40% of the guest molecules 22a depending on the molecular weight of the selected guest molecules 22a. Further, since substantially all of the guest molecules 22a from the starting materials are incorporated into the clathrate compound, there is little or no residual cannabinoid or terpene oil in the final product, which may facilitate further processing and incorporation into tablets.

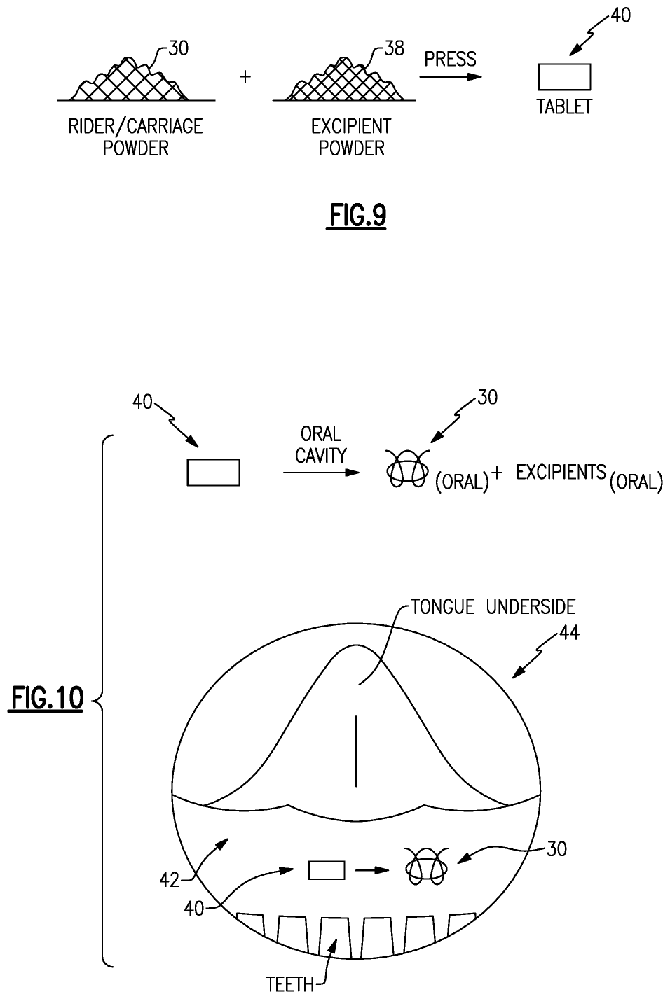

FIG. 8 illustrates that the dry clathrate compound 36 (i.e., clathrate compound 30) is soluble in water or other aqueous solutions. This is possible because the hydrophobicity of the guest molecules 22a resides in the cavities of the carrier molecules 20a. Hydroxyl groups on the carrier molecules 22a, external to the cavity, allow for solvation of the clathrate compound 30 in water.

FIG. 9 illustrates compounding of the clathrate compound 30 with one or more excipients 38 into a tablet 40, such as by using a pill press. Excipients are selected to provide desired characteristic release of the clathrate compound 30. As an example, the excipient or excipients 38 may be selected for fast release in the case of sublingual administration. Excipients for this purpose would include mannitol, polyvinyl alcohol, polyvinypyrrolidone and sterate. In this regard, the transformation of the guest molecules 22a from oil to a powder in the above-described method for producing the clathrate compound 30 is critical for tablet compounding, as oil prevents the formation of a solid, stable tablet.

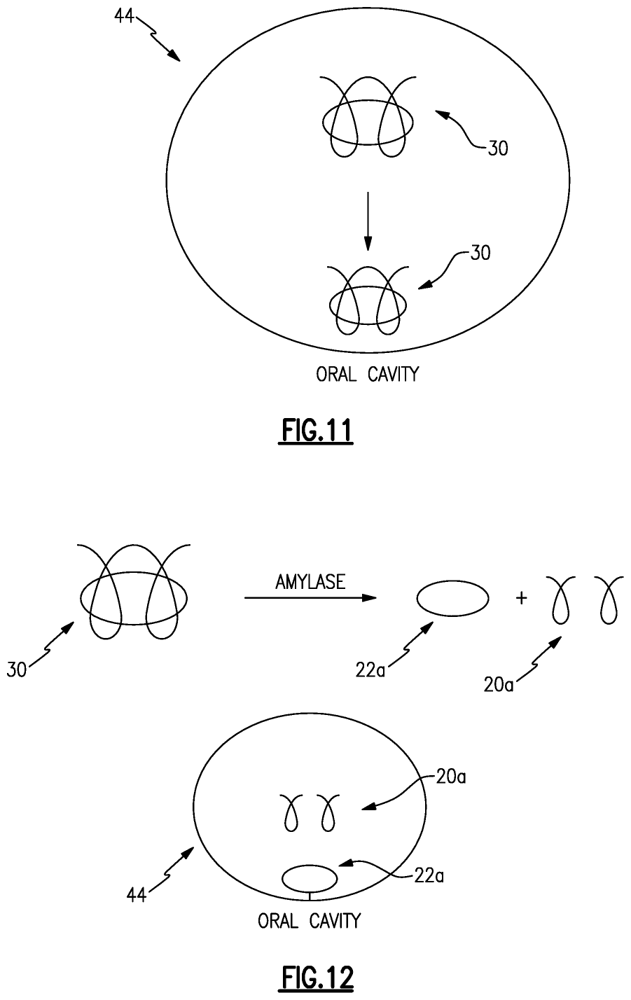

FIG. 10 illustrates the aqueous soluble properties of a tablet 40 that is designed for sublingual release. In an example treatment using the tablet 40, the tablet 40 is placed into the sublingual space 42 in the oral cavity 44. An interaction occurs between the tablet 40 and saliva in the sublingual space 42 which causes rapid tablet disintegration and release of the clathrate compound 30. Release happens based on excipient properties and the water solubility properties of the clathrate compound 30.

In FIG. 11, the release of the clathrate compound 30 into saliva of the oral cavity 44 is shown. As the clathrate compound 30 dissolves in the saliva it moves against the mucosa (sublingual space) and is captured by a process known as mucoadhesion. Mucoadhesion is a bioadhesion process whereby starch-like materials become adsorbed to the mucosa. This results in the clathrate compound 30 being rapidly scrubbed from the saliva and concentrated at the mucosa/saliva interphase. As such, saliva which will eventually be swallowed is substantially devoid of clathrate compound 30. The result is sublingual and very little oral administration of the clathrate compound 30. The treatment may thus include the presentation of the tablet 40 to a subject and/or administration of the tablet 40 into the oral cavity 44 of the subject. Said treatment may further or alternatively include instructions on administration of the tablet 40, including but not limited to instructions on where and how to place the tablet 40 in the oral cavity 44.

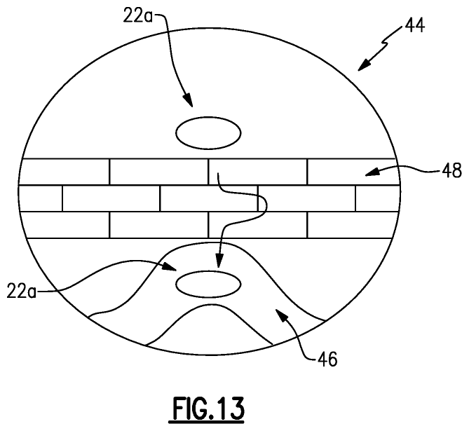

FIG. 12 illustrates a biochemical reaction in which the guest molecule 22a is released from the clathrate compound 30. This reaction occurs when the carrier molecule 20a as part of the clathrate compound 30 becomes a substrate for the enzyme amylase. Cleavage of the clathrate compound 30 into oligosaccharide fragments occurs by a process known as acetolysis. Fragmentation may favor de-adsorption and re-solubilization into saliva leaving the hydrophobic guest molecule 22a bound to the fatty membrane of the mucosa.

FIG. 13 illustrates the transmucosal delivery of a hydrophobic guest molecule 22a to the blood stream 46. The mechanism of delivery is either via extracellular flux through tight cellular junctions as illustrated. Alternatively, delivery may occur intracellularly 48 via transytosis. The latter mechanism may be less efficient as cellular metabolism would limit flux. Regardless, flux of guest molecule 22a toward the blood stream by either mechanism is passive based on a positive gradient.

Although a combination of features is shown in the illustrated examples, not all of them need to be combined to realize the benefits of various embodiments of this disclosure. In other words, a system designed according to an embodiment of this disclosure will not necessarily include all of the features shown in any one of the Figures or all of the portions schematically shown in the Figures. Moreover, selected features of one example embodiment may be combined with selected features of other example embodiments.

The preceding description is exemplary rather than limiting in nature. Variations and modifications to the disclosed examples may become apparent to those skilled in the art that do not necessarily depart from this disclosure. The scope of legal protection given to this disclosure can only be determined by studying the following claims.

What is claimed is:

1. A composition comprising:
a clathrate compound including guest molecules and carrier molecules trapping the guest molecules, wherein the carrier molecules are a saccharide and the guest molecules include at least one of a cannabinoid or a terpene, the clathrate compound including, by combined weight of the carrier molecules and the guest molecules, at least 18% of the guest molecules.

2. The composition as recited in claim 1, wherein the saccharide is cyclic.

3. The composition as recited in claim 1, wherein the saccharide is linear.

4. The composition as recited in claim 1, wherein the carrier molecules include beta cyclodextrin.

5. The composition as recited in claim 1, wherein the carrier molecules include 2-hydroxypropyl-beta-cyclodextrin.

6. The composition as recited in claim 1, wherein the guest molecules include the terpene.

7. The composition as recited in claim 1, wherein the guest molecules include the cannabinoid.

8. The composition as recited in claim 1, wherein the carrier molecules include beta cyclodextrin and the guest molecules include the cannabinoid.

9. The composition as recited in claim 8, wherein cannabinoid is selected from the group consisting of tetrahydrocannabinol, cannabidiol, cannabinol, cannabavarin, cannabigerol, cannabichromene, delta-8-THC, cannabicyclol, cannabitriol, cannabielsoin, and combinations thereof.

10. The composition as recited in claim 1, wherein the clathrate compound includes, by combined weight of the carrier molecules and the guest molecules, up to 40% of the guest molecules.

11. A method of fabricating a clathrate compound, the method comprising:
providing a liquid solution of a first solvent and a second, different solvent;
providing carrier molecules which are soluble in the first solvent and guest molecules which are soluble in the second solvent and insoluble in the first solvent, wherein the carrier molecules are a saccharide and the guest molecules include at least one of a cannabinoid or a terpene;
dissolving in the liquid solution a first amount of the carrier molecules and a second amount of the guest molecules that is at least 1 molar equivalent to the first amount; and
decreasing a concentration of the second solvent in the liquid solution to cause the guest molecules to form a clathrate compound with the carrier molecules in which the carrier molecules trap the guest molecules.

12. The method as recited in claim 11, further comprising, after the decreasing of the concentration, evaporating the liquid solution to produce a dry clathrate compound.

13. The method as recited in claim 11, wherein the second solvent is alcohol.

14. The method as recited in claim 13, wherein the first solvent is water.

15. The method as recited in claim 14, wherein the liquid solution contains, by volume, at least 90% of propanol.

16. The method as recited in claim 15, wherein the carrier molecules include beta cyclodextrin.

17. The method as recited in claim 11, wherein the decreasing of the concentration involves heating the liquid solution to a temperature above the boiling point of the second solvent.

18. A tablet comprising:
one or more excipients; and
a powder containing a clathrate compound including guest molecules and carrier molecules trapping the guest molecules, wherein the carrier molecules are a saccharide and the guest molecules include at least one of a cannabinoid or a terpene, the clathrate compound including, by combined weight of the carrier molecules and the guest molecules, at least 18% of the guest molecules.

19. The tablet as recited in claim 18, wherein the carrier molecules include beta cyclodextrin and the guest molecules include the cannabinoid.

20. The tablet as recited in claim 19, wherein cannabinoid is selected from the group consisting of tetrahydrocannabinol, cannabidiol, cannabinol, cannabavarin, cannabigerol, cannabichromene, delta-8-THC, cannabicyclol, cannabitriol, cannabielsoin, and combinations thereof.
